(19) United States
(12) Patent Application Publication
(10) Pub. No.: US 2024/0373850 A1
(43) Pub. Date: Nov. 14, 2024

(54) Compositions comprising resinous exudate metabolites from plants of the genus Adesmia, used to control and treat infections in plant crops; and methods for controlling infections in plant crops.

(71) Applicants: PONTIFICIA UNIVERSIDAD CATÓLICA DE VALPARAÍSO, Valparaí so (CL); UNIVERSIDAD TÉCNICA FEDERICO SANTA MARÍA, Valparaí so (CL)

(72) Inventors: Rolando Arturo CHAMY MAGGI, Valparaí so (CL); Katy Paulina DÍAZ PERALTA, Viña del Mar (CL); Alejandro MADRID VILLEGAS, Quilpué (CL)

(21) Appl. No.: 18/556,635

(22) PCT Filed: Apr. 22, 2022
(86) PCT No.: PCT/CL2022/050038

§ 371 Date: Oct. 20, 2023

(30) Foreign Application Priority Data
Apr. 22, 2021 (CL) 1035-2021

(51) Int. Cl. A01N 43/16; A01N 35/02; A01P 1/00

(52) U.S. Cl. CPC A01N 43/16; A01N 35/02; A01P 1/00

(57) ABSTRACT

The present invention relates to compositions and methods for controlling infections in plant crops, comprising the application of metabolites to crops infected with various microorganisms, such as microorganisms of the genus Pseudomonas, Erwinia, Bacillus, Botrytis and Phytophthora. The invention also relates to the use of a compound selected from: 2′,4′-dihydroxy-3-(methyl-2-buten-1-yl) dihydrochalcone (1); glabranine (2), isochordine (3), 2′,4′-dihydroxychalcone (4), 7-Hydroxyflavanone (5), chordine (6), izalpinine (7), 2′,4′-dihydroxydihydrochalcone (8), 2′,4′-dimethoxydihydrochalcone (9) and 2′,4′,6′-trihydroxy chalcone (10), in order to prepare an agrochemical composition to control infections in plant crops.

### FIELD OF THE INVENTION

The present invention relates to metabolites of resinous exudates from plants of the genus Adesmia useful for the control and treatment of infections in plant crops and methods for controlling infections in plant crops.

### BACKGROUND OF THE INVENTION

In the state of the art there are various publications that are related to processes to control infections by microorganisms, through the use of compositions from plants of the genus Adesmia. For example, the non-patent document written by A. Agnese, C. Pérez and J. Cabrera, 2001, entitled “Adesmia aegiceras: antimicrobial activity and Chemical study, describes tests on the antimicrobial activity of extracts from the aerial part of the plant Adesmia aegiceras. It was shown that the prepared extracts exhibited antimicrobial activity against Micrococcus luteus, Staphylococcus aureus, Staphylococcus saprophyticus, Escherichia coli, Proteus mirais and Pseudomonas aeruginosa and Candida albicans. The authors use aerial parts of the plant as a substrate, and report an effect of control over fungi and various bacterial species. However, the document does not describe or refer to the effectiveness of the compositions derived from Adesmia on plant pathogens

In the publication by G. Haag, et al., 2014, entitled “Antimicrobial activity of Latin American medicinal plani extracts”, describes extracts from various plants with antimicrobial activity obtained by extraction with dichloromethane and ethanol. One of the species used is Adesmia bicolor, however, it turned out not to have an antimicrobial effect against Pseudomonas.

In the publication González SB (2005) Adesmia boronioides hook. f.: an aromatic and medicinal species native to Patagonia. Patagonian Naturalia 2 (1): 85-91; The antimicrobial and antifungal activity test of compositions derived from Adesmia boronoides, Hook, is reported, for which material from the aerial part of the plant (secondary branches and leaves) was used as substrate, and extracts were prepared in methanol, acetate ethyl, dichloromethane, hexane and water. However, this publication is silent regarding the potential use of these compositions in the control of plant pathogens, nor is a use concentration established.

In the publication Blengini A A, et al (2016) Evaluation of the antimicrobial activity of paramela (Adesmia boronioides, Hook). Dominguezia 32 (2): 25 describes the obtaining of an essential oil of Adesmia boronioides by hydrodistillation of the aerial parts of said plant species, which was tested on a series of gram positive and negative bacteria. The essential oil of Adesmia boronioides showed antimicrobial activity, especially against Staphylococcus aureus. However, this publication is silent regarding the potential use of these compositions in the control of plant pathogens, nor is a use concentration established. Finally, Chilean patent application 0881-2018 describes a procedure for preparing a composition of resinous exudates from the aerial parts of plants of the genus Adesmia, to control in vitro and in vivo infections in plant cultures, comprising: obtaining and selecting aerial parts of plants of the genus Adesmia; cut the aerial parts of the selected plants; immerse the cut plant material in a solution containing 1,3-dioxolane, or dichloromethane; filter the solution from the previous step and extract all the liquid solvent; separate the bagasse obtained from the used filter; and concentrating the plant exudate, such as those described in the present invention. In addition, methods are described to control infections in plant cultures, which comprise applying the composition obtained by means of the described procedure to cultures infected with various microorganisms, such as microorganisms of the genus Pseudomonas, Erwinia, Bacilius, Staphylococcus, Listeria, Botrytis and Phytophthora. In any case, this patent does not specify the active compounds or secondary metabolites that generate the antibacterial activity.

However, none of the documents of the state of the art mentions a composition comprising the secondary metabolites described in the present invention to control infections in plant crops, preferably Pseudomnas syringae pv actinidiae (Psa) in Kiwi crops. The metabolites of the invention show an antibacterial activity from 50 to 400 pg/mL in vitro and compound 4 shows an in vivo inhibition of the disease caused by Psa in Kiwi cultures at a concentration of 500 ug/mL.

### BRIEF DESCRIPTION OF THE INVENTION

Methods for controlling infections in plant cultures are described, which comprise applying the metabolites to cultures infected with various microorganisms, such as microorganisms of the genus Pseudomonas, Erwinia and Bacillus, which affect plant crops. The secondary metabolites identified in the present invention obtained from the resinous exudate of A. balsamica and A. resinosa have antibacterial effects in vitro, and are not toxic.

### BRIEF DESCRIPTION OF THE FIGURES

FIG. 1: Spectra of Molecule 1. A) FT-IR; B) ¹H NMR; C) ¹³C NMR; D) MS.

FIG. 2: Spectra of Molecule 2. A) FT-IR; B) ¹H NMR; C) ¹³C NMR; D) MS.

FIG. 3: Spectra of Molecule 3. A) FT-IR; B) ¹H NMR; C) ¹³C NMR; D) MS.

FIG. 4: Spectra of Molecule 4. A) FT-IR; B) ¹H NMR; C) ¹³C NMR; D) MS.

FIG. 5: Spectra of Molecule 5. A) FT-IR; B) ¹H NMR; C) ¹³C NMR; D) MS.

FIG. 6: Spectra of Molecule 6. A) FT-IR; B) ¹H NMR; C) ¹³C NMR; D) MS.

FIG. 7: Spectra of Molecule 7. A) FT-IR; B) ¹H NMR; C) ¹³C NMR; D) MS.

FIG. 8: Spectra of Molecule 8. A) FT-IR; B) ¹H NMR; C) ¹³C NMR; D) MS.

FIG. 9: Spectra of Molecule 9. A) FT-IR; B) ¹H NMR; C) ¹³C NMR; D) MS.

FIG. 10: Spectra of Molecule 10. A) FT-IR; B) ¹H NMR; C) ¹³C NMR.

FIG. 11: Effect of 2′, 4′ dihydroxychalcone on the integrity of the cell membrane of E. carotovora. The bacteria were incubated in a culture medium at 22 QC in the presence of 30% (v/v) ethanol (a), 5% methanol; 50 ug/mL of the compound against E. carotovora seen in bright field (b) and seen under fluorescence (c) for 3 hours of incubation.

### DETAILED DESCRIPTION OF THE INVENTION

The metabolites object of the present invention are obtained from plant exudates of the genus Adesmia for the control of agricultural and clinical pathogens of economic importance under in vitro and in vivo conditions.

Adesmia exudates have hydrophobic properties that can be obtained by a standard 1,3-dioxolane immersion of the aerial parts. Semi-polar extraction can be followed by a filtration and concentration process. The exudate has in common with the biopesticides that are normally used to control the disease in Kiwi plantations its antimicrobial effects in vitro, but it differs from many of them due to its lack of toxicity, being a natural exudate, maintaining the aforementioned effect, which confer efficiency in the control and safety to the environment.

The exudate can be obtained by immersion of leaves and bark of Adesmia. Other solvents can be used in substitution of 1,3-dioxolane such as dioxane, dichloromethane or other semi-polar ones, without limiting the application of other solutions. After immersion it is possible to isolate it or the active compounds. After evaporation of volatile solvents, the exudate is typically syrupy in consistency and reddish-brown in color. Both consistency and color characteristics may vary depending on whether the exudate is diluted with ethanol or other solvents.

The process of preparing the composition to control infections in plant crops based on exudates from plants of the genus Adesmia, comprises the following stages:
i. Stage 1: Consists of obtaining the raw material from which the exudates are obtained. The plant material of the aerial part that is collected is manually selected; it is used fresh and clean.
ii. Stage 2. It consists of the immersion of the vegetative material. The immersion process consists of placing the cut plant material in a 50 kg capacity stainless steel container with the solvent 1,3-dioxolane or dichloromethane. Once the plant material is introduced, it is submerged for a period of 35 to 45 seconds.
iii. Stage 3. It consists of filtering the immersion by decantation with a paper filter of 11 pm pore size, in order to remove all the plant material that may fall into the container and traces of dust and earth.
iv. Stage 4. It consists of the separation of the bagasse from the filter once all the liquid has been extracted.
v. Stage 5. It consists of the concentration of the exudate. Once the exudates are obtained, the solution resulting from the filtration of the immersion in a balloon is emptied to concentrate in a rotary evaporator that operates under the following conditions: Water bath at a temperature of 40° C. and a pressure of 0.1 bar vacuum.

Final stage. It consists of obtaining and storing the concentrate obtained in amber containers and stored at a temperature of 4° C.

### Composition of Resinous Exudate of A. balsamica

Five main secondary metabolites were identified in the exudate of Adesmia balsamica, ordered by increasing polarity (Table 1).

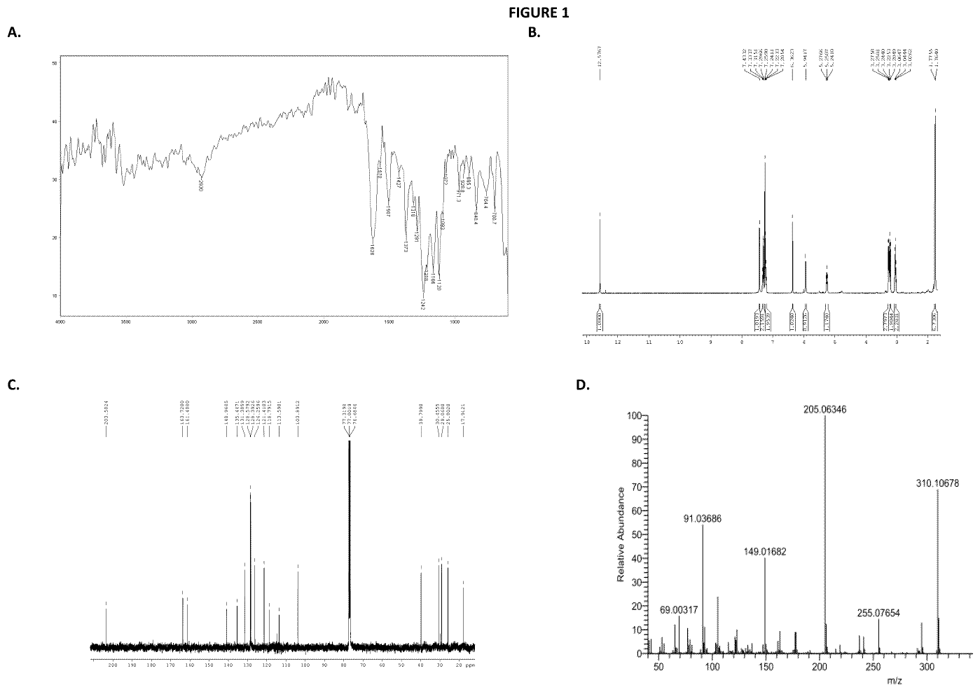

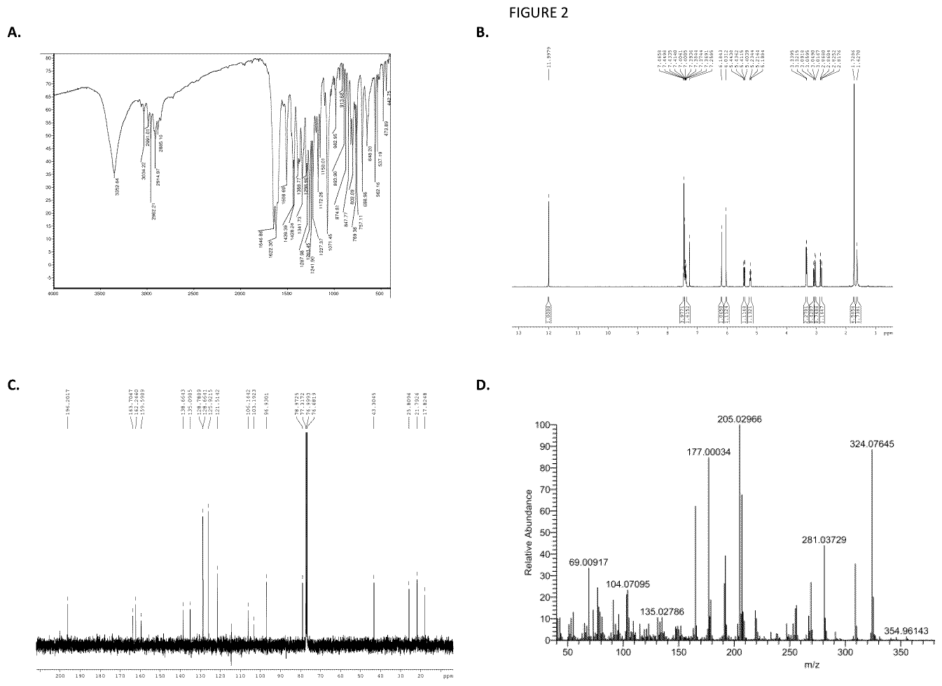

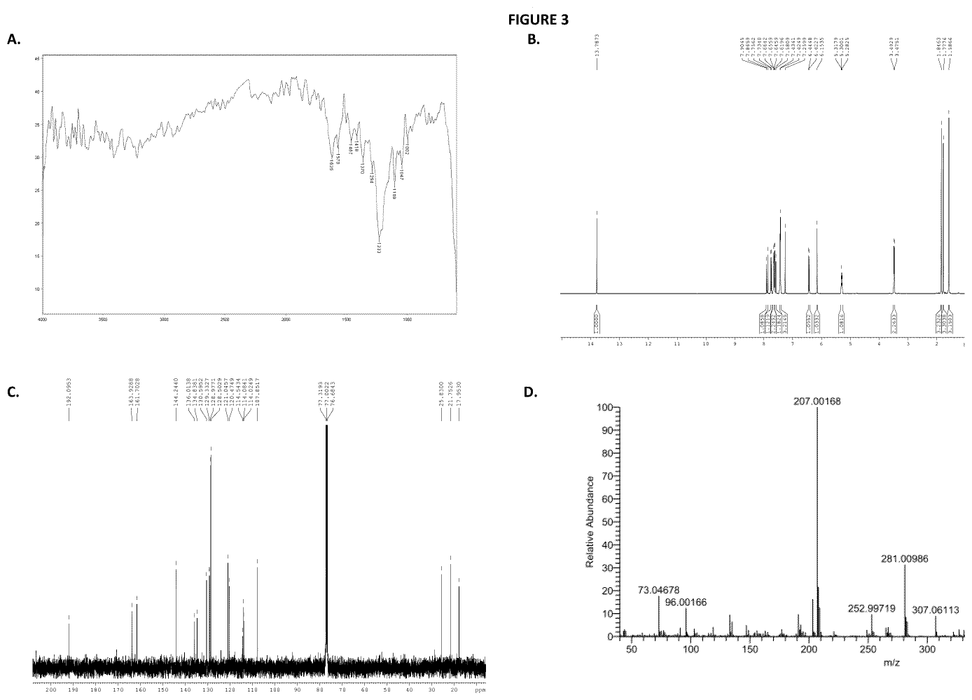

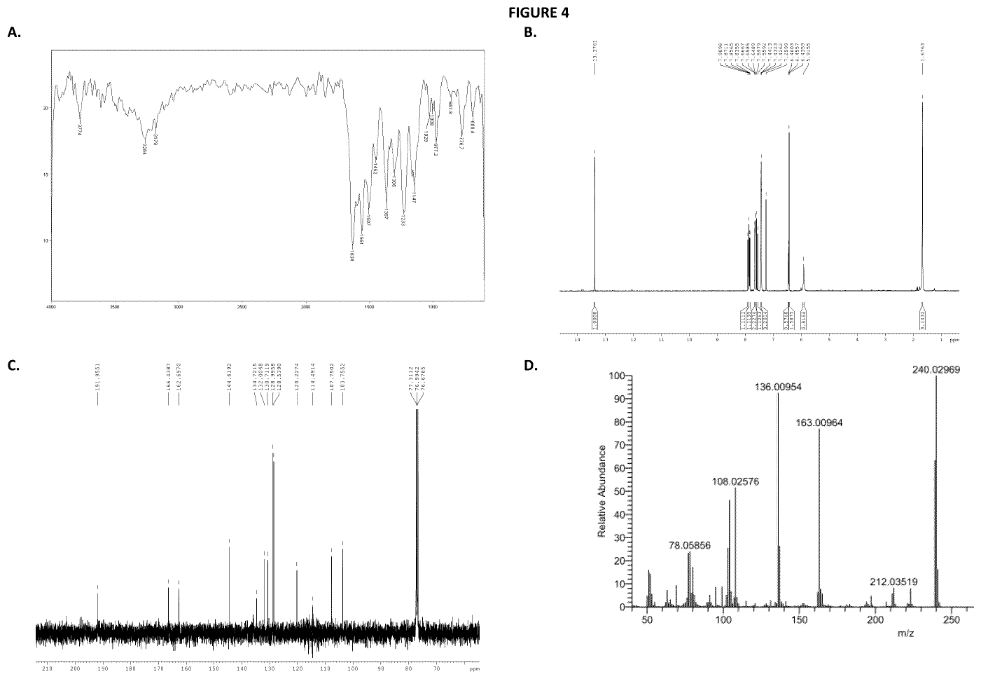

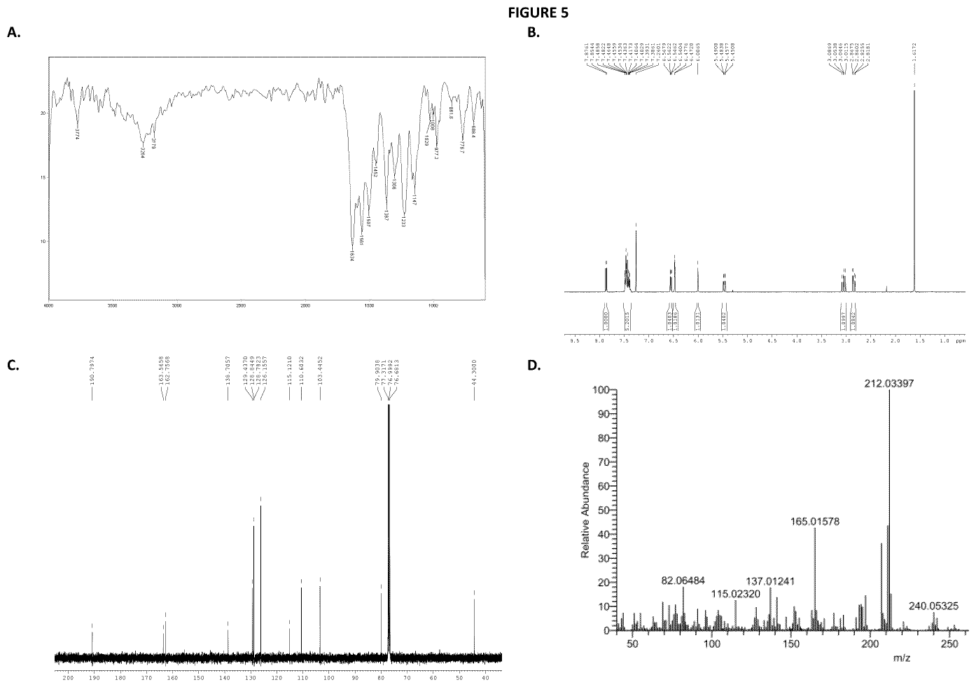

All compounds are known 2′,4′-dihydroxy-3-(methyl-2-buten-1-yl) dihydrochalcone (1), glabranin (2), isochordine (3), 2′,4′-dihydroxychalcone (4) and 7-Hydroxyflavanone (5). The compounds correspond to a prenylated dihydrochalcone, a prenylated flavanone, a prenylated chalcone, a chalcone and a flavonone, respectively. The structural determination of compounds 1-5 was carried out using NMR (FIGS. 1 to 5), and confirmed by comparison of the physical and spectroscopic properties of the isolated compounds against literature data [1-5]

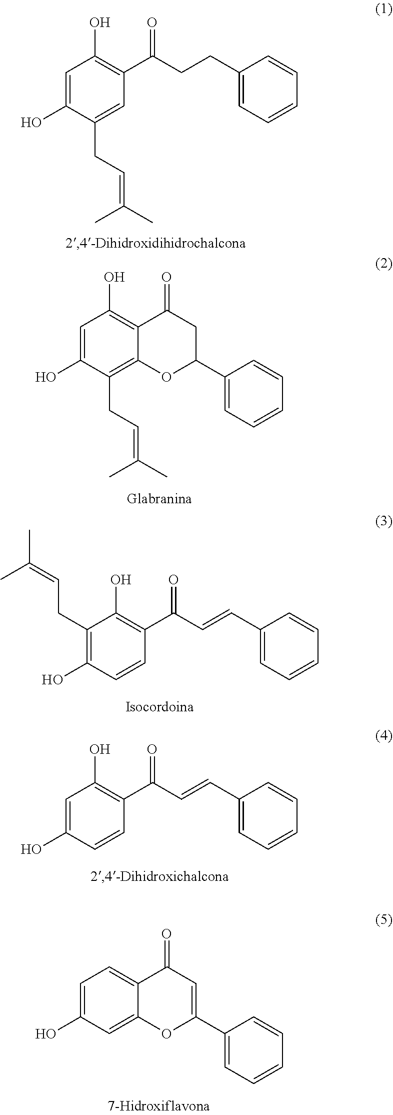 [SMILES: CC(C)=CCc1cc(C(=O)CCc2ccccc2)c(O)cc1O ; CC(C)=CCc1c(O)cc(O)c2c1OC(c1ccccc1)CC2=O ; CC(C)=CCc1c(O)ccc(C(=O)/C=C/c2ccccc2)c1O ; O=C(/C=C/c1ccccc1)c1ccc(O)cc1O ; O=c1cc(-c2ccccc2)oc2cc(O)ccc12]

Structures of secondary metabolites present in the exudate of A. balsamica (1-5).

TABLE 1
Contribution of secondary major metabolites isolated from the
resinous exudate of A. baslamica by column chromatography.

| Compounds | Mass (g) | Yield (%) |
|---|---|---|
| 1 | 0.0585 | 0.39 |
| 2 | 0.315 | 2.1 |
| 3 | 0.12 | 0.8 |
| 4 | 1.31 | 8.7 |
| 5 | 0.045 | 0.3 |

*Total mass: 15 grams of extracted exudate (100%).

The main compound isolated from the resinous exudate of A. balsamica is the 2′,4′-dihydroxychalcone compound (4).

In one of the modalities of the present invention, a composition is described to control bacterial infections, which comprises as an active ingredient an effective amount between 50 mg/L to 1000 mg/L, of a compound selected from:
2′,4′-dihydroxy-3-(methyl-2-buten-1-yl) dihydrochalcone (1);
glabranin (2);
isochordine (3);
2′,4′-dihydroxychalcone (4);
7-Hydroxyflavanone (5);
chordine (6);
izalpinine (7);
2′,4′-dihydroxydihydrochalcone (8);
2′,4′-dimethoxydihydrochalcone (9); and
2′,4′,6′-trihydroxy chalcone (10);

wherein said composition further comprises an agronomically acceptable carrier. In one of the modalities of the present invention, a method for controlling bacterial infections is described, which comprises applying an effective amount between 50 mg/L to 1000 mg/L, of the composition described in claim 1 to the bacteria to be treated, wherein said composition has antibacterial activity under in vitro and in vivo conditions.

In one of the embodiments of the present invention, the use of a compound selected from: 2′,4′-dihydroxy-3-(methyl-2-buten-1-yl) dihydrochalcone (1), glabranin (2), isochordine (3), 2′,4′-dihydroxychalcone (4), 7-Hydroxyflavanone (5), chordine (6), izalpinin (7), 2′,4′-dihydroxydihydrochalcone (8), 2′, 4′-dimethoxydihydrochalcone (9) and 2′,4′,6′-trihydroxy chalcone (10), which is used to prepare an agrochemical composition to control infections in plant crops and which comprises an effective amount between 50 mg/L to 1000 mg/L, of said compound. In one of the embodiments of the present invention, it is described that the infections are caused by microorganisms of the species Pseudomonas syringae pv actinidiae code 770 and 8.43 and other agronomically relevant bacteria.

### EXAMPLES

### Composition of Resinous Exudate of A. resinosa

Eight main secondary metabolites were identified in the resinous exudate of A. resinosa (Table 2). Three were previously isolated from the resinous exudate of A. balsamica, glabranin (2), isochordine (3), 2′,4′-dihydroxychalcone (4).

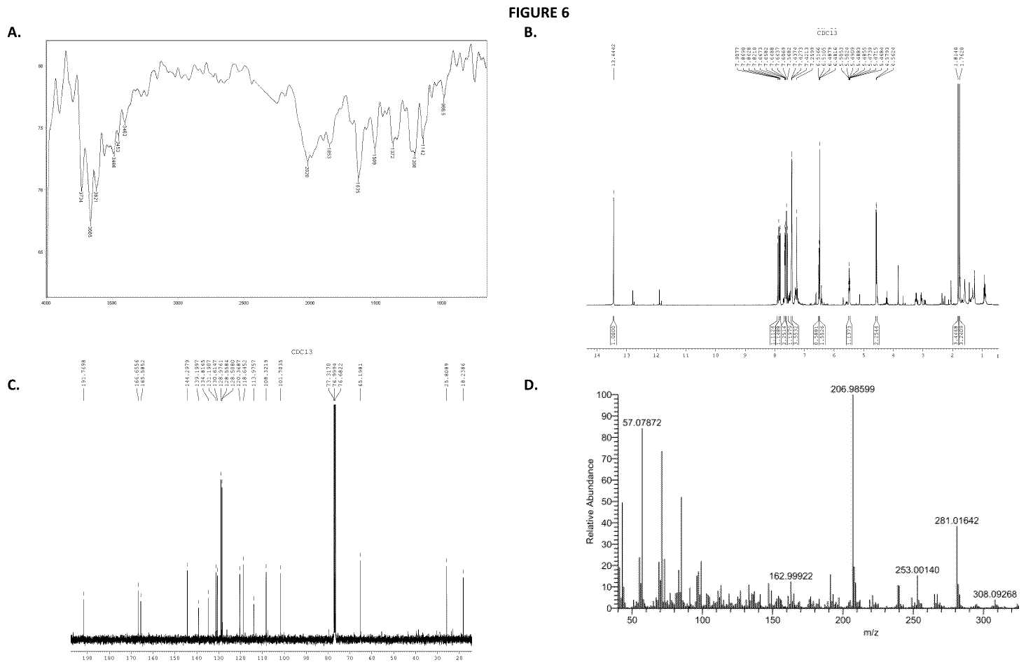

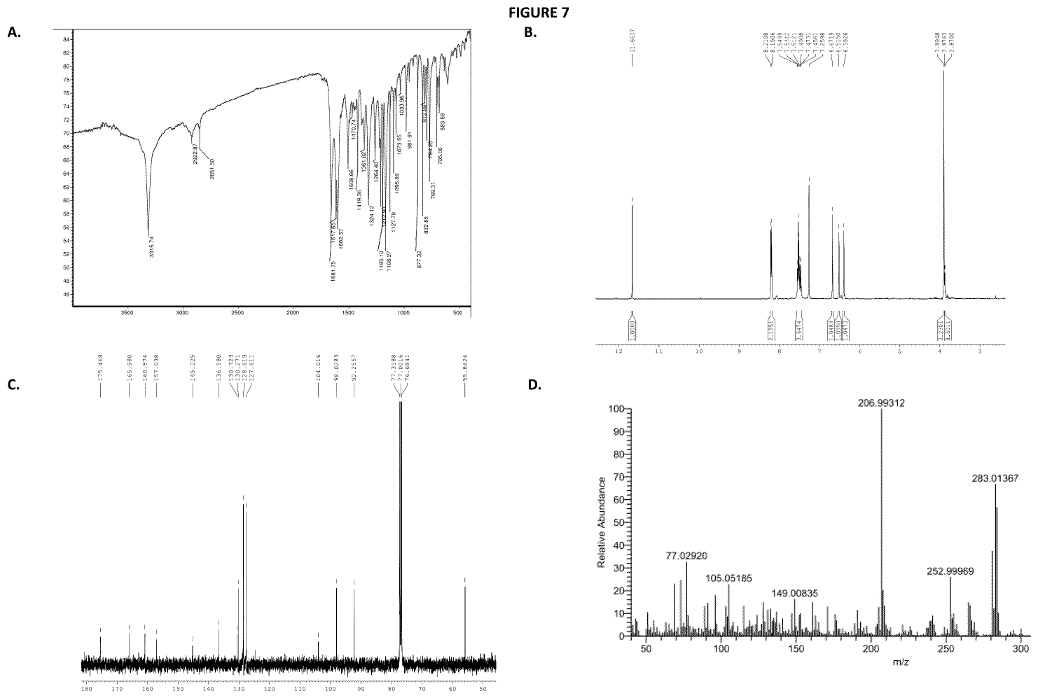

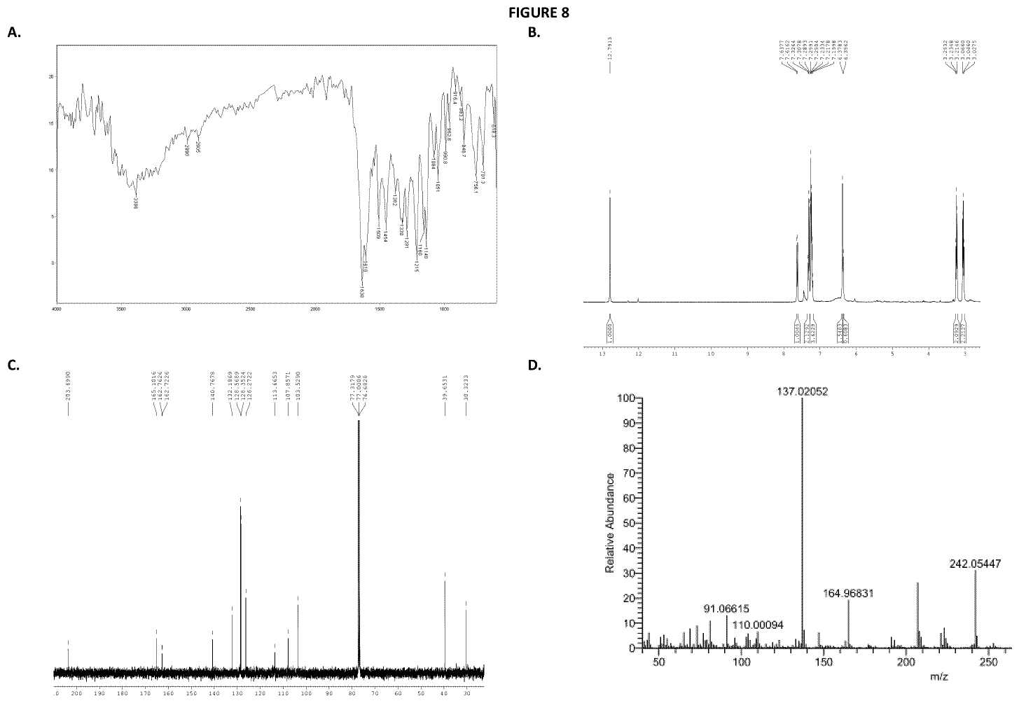

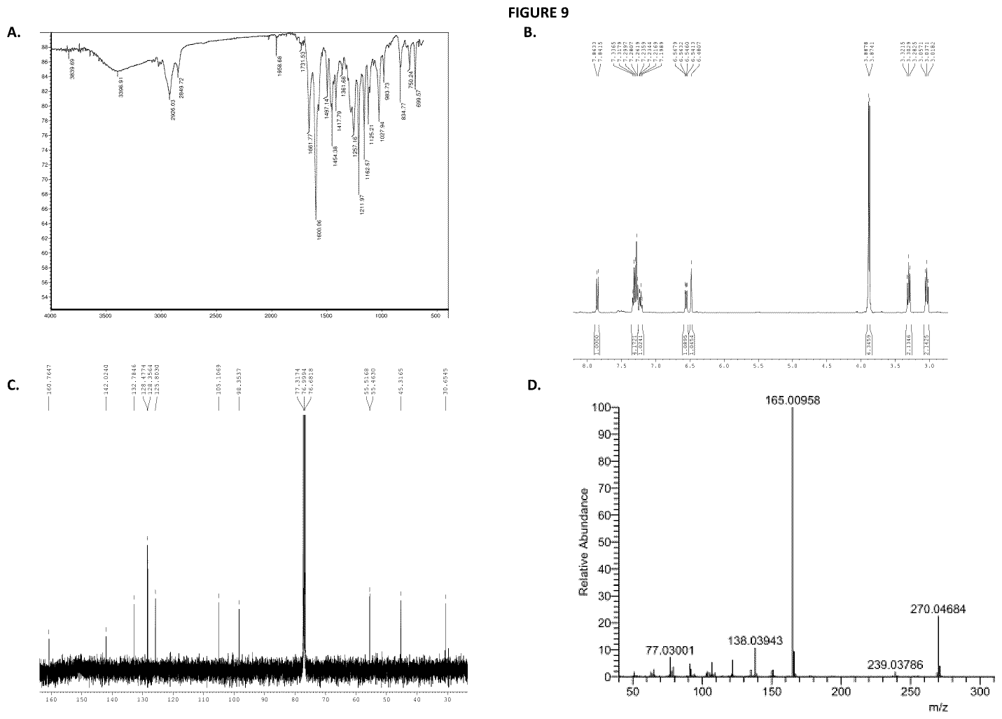

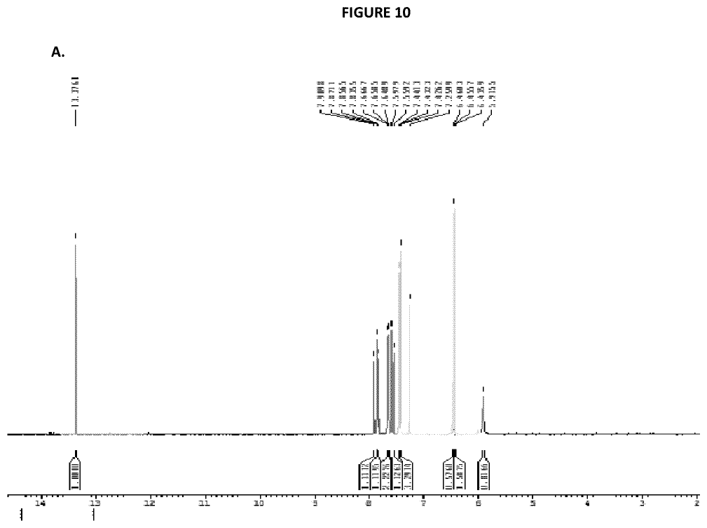

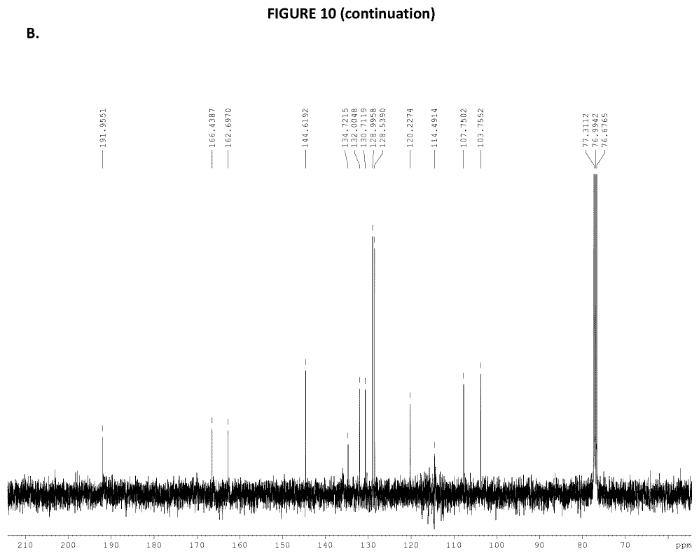

The following five secondary metabolites were identified as the known compounds, chordine (6), izalpinin (7), 2′,4′-dihydroxydihydrochalcone (8), 2′,4′-dimethoxydihydrochalcone (9) and 2′,4′, 6′-trihydroxy chalcone (10). These compounds correspond to an oxyprenylated chalcone, a flavone, a dihydrochalcone, an oxyalkyldihydrochalcone and a trihydroxychalcone, respectively. The structural determination of compounds 6-10 was carried out using NMR (see FIGS. 6-10), and confirmed by comparison of the physical and spectroscopic properties of the isolated compounds against literature data [2-8].

The eight isolated compounds belong to the flavonoid family and present the same particular characteristic of non-substitution of the B ring as the metabolites found in the resinous exudate of A. balsamica, with a great structural difference in the substituents found in the aromatic ring A such as the presence of prenoxy groups in compound 6 and methoxy groups in compounds 7 and 9.

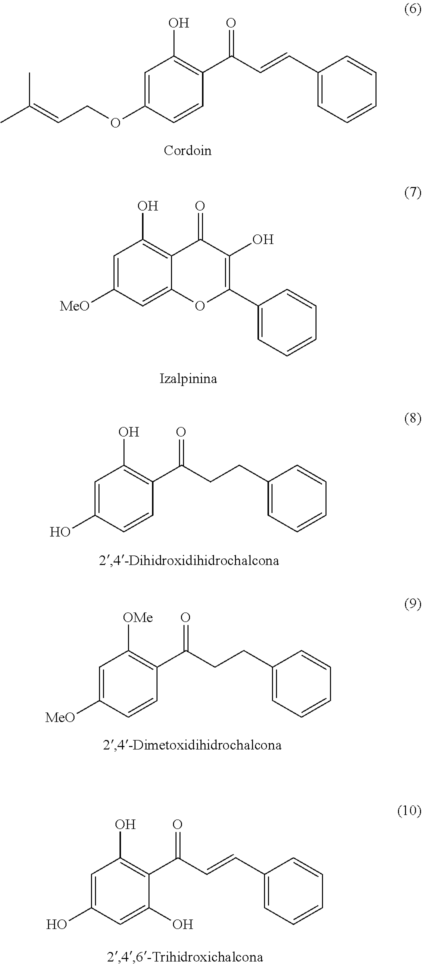 [SMILES: CC(C)=CCOc1ccc(C(=O)/C=C/c2ccccc2)c(O)c1 ; COc1cc(O)c2c(=O)c(O)c(-c3ccccc3)oc2c1 ; O=C(CCc1ccccc1)c1ccc(O)cc1O ; COc1ccc(C(=O)CCc2ccccc2)c(OC)c1 ; O=C(/C=C/c1ccccc1)c1c(O)cc(O)cc1O]

Structures of secondary metabolites present in the exudate of A. resinosa (6-10).

TABLE 2
Contribution of major secondary metabolites isolated from the
resinous exudate of A. resinosa by column chromatography.

| Compounds (g) | Mass (g) | Yield (%) |
|---|---|---|
| 2 | 0.255 | 1.7 |
| 3 | 0.195 | 1.3 |
| 4 | 1.50 | 10 |
| 6 | 0.015 | 0.1 |
| 7 | 0.012 | 0.08 |
| 8 | 0.195 | 1.3 |
| 9 | 0.345 | 2.3 |
| 10 | 0.475 | 3.2 |

* Total mass: 15 grams of extracted exudate (100%).

The main compound isolated from the resinous exudate of A. resinosa is the 2′,4′-dihydroxychalcone compound (4).

### Assay Bacterial Strains and Antibacterial Test (MIC)

Antibacterial test of secondary metabolites isolated from the resinous exudates of Adesmia plants against the pathogen (Psa), from isolates made in Chile and Italy, also tested on other bacteria of interest, in vitro.

All strains were cultivated in Nutrient culture medium supplemented with 3% sucrose (SNA), and incubated at 27° C. The bacterial inoculum was produced in Mueller-Hinton (MH) culture medium in glass test tubes and incubated at 27° C. in an orbital shaker at 150 rpm in the dark for 24 hours. A stock solution of each molecule was prepared to obtain a concentration of 2000 mg/L, later they were diluted in a 96-well microplate with MH culture medium and thus obtain the different concentrations of 1, 25, 50, 100, 200, 400, 800 and 1600 pg/mL of each molecule. The antibacterial activity of the molecules was determined by the method of micro-dilution of the substance to be tested. Four controls were used: negative control (only MH liquid culture medium inoculated with bacteria); positive control (liquid MH culture medium adding Chloramphenicol at the same concentrations tested inoculated with bacteria and in the case of the Italian strain, copper sulfate was added), negative control (1) only substances plus solvent (liquid MH culture medium plus ethanol at 1% without inoculating bacteria) and a white control (only MH liquid culture medium, without inoculating). The bacterial suspension of each of the strains was adjusted to 0.5 McFarland (1×10⁸ cfu/ml), inoculating 1.5 μL per well in the microplate and subsequently incubated at 27° C. for 24 hours.

Through the evaluation of absorbance at 600 nm in an Accu Reader M965 spectrophotometer, those treatments that were effective in inhibiting growth compared to the negative control were selected, to subsequently quantify the percentage of inhibition. The absorbances or optical density (O.D) were converted to percentage of antibacterial inhibition (PIAB) using the following formula:

$$PIAB = 100 * ( ( OD C . Negative - OD Substance ) / ( OD C . Negative - OD C . Positive ) )$$

In addition, 100 mL aliquots of each of the concentrations were swept in a petri dish to observe the presence or absence of Psa colonies after 48 hours.

TABLE 3
Minimum Inhibitory Concentration MIC (pg/mL) of pure isolated
compounds against different bacterial strains.

| | Bacterial strains MIC (μg/mL) | | | | |
|---|---|---|---|---|---|
| Compound | Psa 770* | Psa 8.43* | Pss* | E.c* | B.s* |
| 1 | ≥400 | ≥200 | — | — | — |
| 2 | ≥400 | — | — | — | — |
| 3 | ≥1600 | — | — | — | — |
| 4 | ≥200 | ≥50 | — | ≥10 | ≥10 |
| 5 | ≥200 | ≥100 | — | | |
| 9 | ≥400 | ≥1600 | ≥50 | ≥10 | ≥10 |
| 10 | ≥50 | ≥50 | ≥50 | | ≥10 |
| 11 | ≥200 | — | — | — | — |

*Psa: Pseudomonas syringae pv actinidiae code 770 and 8.43; Pss: Pseudomonas syringae syringae; Ec: Erwinia carotovora; Bs: Bacillus subtilis; —: they were not tested.

Determination of the effect of compound 2 4′ dihydroxychalcone (compound No. 4) on the integrity of the membrane of Gram negative and Gram positive bacteria.

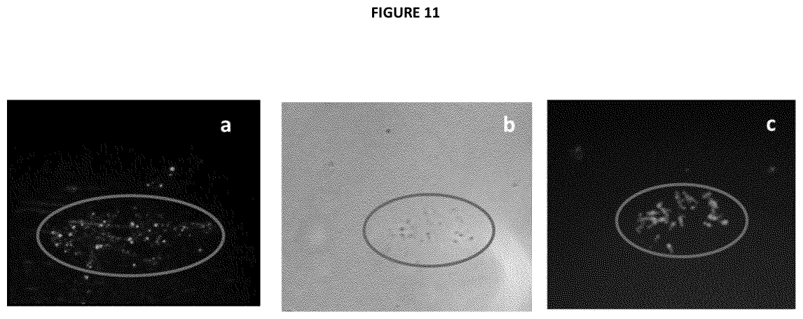

Because several studies have established that phenolic compounds destroy the bacterial membrane, the effect of the major compound 2′, A′ dihydroxychalcone (compound No. 4) present in the exudate of plants of the Adesmia genus was evaluated, which was attributes the antibacterial activity that they present against Pseudomonas syringae pv actinidiae (Psa). It was tested against Gram-negative bacteria of agricultural importance Erwinia carotovora by Sytox Green staining. It was observed that the negative control (5% methanol) exhibits nuclei without fluorescence emission, indicating that there is no alteration of the membrane, while the positive control (30% ethanol) exhibits bacterial nuclei with fluorescence, clearly indicating alteration of the membrane. membrane integrity (FIG. 11a and b). On the other hand, when treating the bacteria S. aureus and E. carotovora with 50 ug/mL of the compound, the alteration of the plasmatic membrane was observed in both bacteria after 3 hours of incubation. In this way, through the observation of nuclei stained with Sytox in the fluorescence microscope (100×), it is evident that the mechanism of action of the compound against both the Gram positive bacteria S. aureus and Gram negative E. carotovora is the destruction of the plasma membrane (FIG. 11c). Therefore, it is demonstrated that the mechanism of action that this type of exudate, extracts and compound also uses against Pseudomonas syringae pv actinidiae (Psa) is contact, breaking the cell membrane and leaving the nucleus exposed.

In vivo antibacterial activity tests of the compounds of the present invention, in Kiwi crops.

It is important to point out that the Chilean patent application 0881-2018 of the same inventors of the present invention, describes a procedure to prepare a composition obtained from resinous exudates of the aerial parts of plants of the genus Adesmia, to control in vitro and in vivo infections. in plant crops. In addition, methods for controlling infections in plant crops are described, which comprise applying said composition to crops infected with various microorganisms. In any case, this patent did not specify the active compounds or secondary metabolites that generate the antibacterial activity, and which are the compounds that have been characterized later and that describe the secondary metabolites of the present invention. For this, Kiwi A. chinensis seedlings from in vitro propagation were used. The bacterium Pseudomonas syringae pv actinidiae (Psa) 190770 used in the in vitro tests was used. The plants were kept in aseptic chambers with 50% relative humidity, under controlled temperature conditions (20° C.±2) and a photoperiod regimen of 12 hours of light and 12 hours of darkness. Both the application of the treatments and inoculation were applied in the summer season. The bacterial inoculum was sprayed on the surface of the leaves of the plants, which had been subjected to small wounds on the leaves. The plants were sprayed with different treatments (Table No. 5, first column) 24 hours before inoculation. Control plants were only sprayed with sterile distilled water and kept isolated from the others treated with Psa.

The evaluations were made periodically to observe the symptoms of the plant (Table No. 4) and the samples were taken at the third and fourth week from the inoculation, in which the leaves were collected at the moment of beginning to see symptoms or in their defect a month after starting the trial, they were frozen in RNALater® and stored at −80° C. for RNA isolation.

TABLE NO. 4
Scale or levels of damage of the symptomatology
product of the Psa Infection.

| Note | Description | Category |
|---|---|---|
| 0 | Plant without presence of symptoms (spots on leaves) | Fury |
| 1 | Plant with 1 leaf with spots | Mild |
| 2 | Plants with 2 leaves with spots | Moderate |
| 3 | Plants with more than 3 leaves with spots and in some | Severe |
| | cases without leaves | |

From the treatments listed in Table No. 5, it was possible to show that the lesions were small at the beginning, but they progressed as the days passed. The application of all the treatments was carried out at a concentration of 500 ug/mL, not observing any type of phytotoxicity.

TABLE NO. 5
Percentage of incidence of total damage and Incidence
values according to the scale category generated for
the disease in the different in vivo treatments.

| | | Level of |
|---|---|---|
| | | Incidence |
| | Percentage | according |
| | of | to category |
| | incidence | (Maximum |
| Treatment | (%) | value 3) |
| T1 (Water without inoculum is applied) | 0 | 0 |
| T2 (Water + Psa inoculum is applied) | 100 | 2.6 |
| T3 (The compound 2′,4′ dihydroxychalcone + | 40 | 0.4 |
| Psa inoculum is applied) | | |
| T5 (A. balsamica exudate from application CL | 80 | 1.2 |
| 0881-2018 + Psa inoculum is applied) | | |
| T6 (A. resinosa exudate from application CL | 60 | 0.8 |
| 0881-2018 + Psa inoculum is applied) | | |
| T10 (Commercial Bactericide + Psa inoculum | 0 | 0 |
| is applied) | | |
| T11 (The bacteria is applied before and after | 100 | 2.8 |
| having applied the A. balsamica Effective | | |
| Exudate formulation (Healing Treatment). | | |

According to the treatments that were analyzed phytopathologically, it can be concluded that the preventive treatment with 2′,4′ dihydroxychalcone (Treatment 3) was the one that behaved best, presenting 60% of plants categorized at level 0, that is, plants that they did not present visual symptoms of the disease. In addition, the preventive treatment with 2′,4′ dihydroxychalcone (Treatment 3), was significantly better compared to the treatments carried out with the compositions disclosed in patent application CL 0881-2018, since the resinous exudate from A. resinosa (treatment 6) presented 40% of healthy plants; and the one based on exudate from A. balsamica (treatment 5) presented 20% of plants without affection.

All these emulsions were applied by spraying on the plant before and after inoculating the bacteria, where it was possible to show that the plants continued to grow without problems, they even sprouted and their leaves grew without showing symptoms of the disease. It should be noted that this disease, being asymptomatic, the visual results are not conclusive to establish which emulsion was the most effective, which is why molecular analyzes are required.

For this, the total RNA of the problem samples was isolated from 80 to 100 mg of tissue from the leaves of A. chinensis belonging to the applied treatments, which were extracted with the E.Z.N.A.® PLANT RNA Kit using the protocol for difficult samples described by the manufacturer and the concentration of the extracted material was quantified through the Nanodrop equipment. The qRT-PCR was performed using the AriaMx Realtime PCR System equipment, using the Takion Sybrgreen+One step converter kit and specific primers synthesized by IDT (Integrated DNA Technologies) (See Table 6).

TABLE NO 6
Primers used in this study for the detection of
Psa using real-time qPCR.

| Primer | | Amplification |
|---|---|---|
| reference | Primer Sequence | size |
| Wth-intmod-F | ATGTTCGAGCTGGCTGAAG | 119 |
| Wth-intmod-R | GCGTGCACGTTGTCAATG | |

The program conditions were 48° C. for 10 min, 95° C. for 3 min, followed by 40 cycles of 95° C. for 10 sec, alignment and extension at 60° C. for 50 sec; then the melting curves 95° C. for 30 sec., 65° C. for 30 sec., and 95° C. for 30 sec. The linear regression curve was constructed by the team and established in the graph made based on the Ct of each reaction against the logarithmic value of Psa DNA concentrations. The slope of the standard curve that was generated for each PCR run was used in the following equation to estimate the reaction efficiency (E) where E optimal=2 which corresponds to 100% efficiency. The number of copies/ng of Total Psa RNA were calculated through the equation of the straight line provided by the equipment, which was analyzed with the standard curve included in each qRT-PCR run.

It was found that the samples belonging to treatment 1 (negative control to which only water was applied) did not amplify with the selected primer, therefore, the acquired plants did not present Psa in their leaf tissue and were not contaminated during the course of the test.

Treatment 2 (water, inoculated with Psa) was the only one that presented samples infested with Psa, giving a Tm of 85, which corroborates that the inoculation was applied properly.

In addition, in treatment 10, which corresponds to the application of the copper-based Nordox® pesticide that is currently applied to kiwi crops at 1.3-2 g/L, it protected the plants from the bacteria, preventing the development of the disease or that the plants presented some type of symptomatology. none of the samples analyzed amplified the Tm of the Psa blank (Table N Q 7), so the use of these emulsions as preventive control of kiwi bacteriosis are effective when applied before and after the bacteria is inoculated on the leaves of the floors.

TABLE NO. 7
Absolute quantification of Psa infecting kIwI plants
obtained by qRT-PCR 3-4 weeks after inoculation.

| | | Tm | Number | DNA | Copies/ng |
|---|---|---|---|---|---|
| Treat- | | product | of DNA | concentration | of total |
| ment | Ct | (−R′(T)) | copies | (ng/μL) | RNA |
| 1 | No Cq | 0.0 | — | — | — |
| | No Cq | 0.0 | | | |
| | No Cq | 0.0 | | | |
| 2 | 19.40 | 84 | 1.20*10¹⁰ | 77.87 | 1.54*10⁸ |
| | 19.46 | 84.5 | 1.14*10¹⁰ | 77.87 | 1.46*10⁸ |
| | 19.41 | 84.5 | 1.19*10¹⁰ | 77.87 | 1.53*10⁸ |
| 3 | 29.17 | 81.5 | 4.67*10⁶ | 24.16 | 1.93*10⁵ |
| | 27.80 | 84 | 1.40*10⁷ | 24.16 | 5.79*10⁵ |
| | 30.01 | 82 | 2.38*10⁶ | 24.16 | 9.85*10⁴ |
| 5 | 27.66 | 84 | 1.57*10⁷ | 29.83 | 5.26*10⁵ |
| | 29.21 | 81.5 | 4.54*10 | 29.83 | 1.52*10⁵ |
| | 29.18 | 81.5 | 4.64*10⁷ | 29.83 | 1.56*10⁵ |
| 6 | No Cq | 0.0 | | | |
| | No Cq | 0.0 | | | |
| | No Cq | 0.0 | | | |
| 10 | No Cq | 0.0 | — | — | — |
| | No Cq | 0.0 | | | |
| | No Cq | 0.0 | | | |
| Control | 13.29 | 84.5 | N/C | N/C | N/C |
| | 13.29 | 84.5 | | | |
| | 13.29 | 84.5 | | | |
| Control | No Cq | 0.0 | — | — | — |
| | No Cq | 0.0 | | | |
| | No Cq | 0.0 | | | |

N/C: Not quantified.

Therefore, it can be concluded that the preventive treatment with 2′,4′ dihydroxychalcone (Treatment 3) was the one that obtained the best results in the analyzes phytopathological and its confirmation by qRT-PCRT, since 60% of plants were observed that did not present visual symptoms of the disease.

In addition, the phytopathological analyzes and their confirmation by qRT-PCRT, revealed that the preventive treatment with 2′, 4′ dihydroxychalcone (Treatment 3), was significantly better compared to the treatments carried out with the compositions disclosed in patent application CL 0881-2018, since the resinous exudate from A. resinosa (treatment 6) presented 40% of healthy plants; and the one based on exudate from A. balsamica (treatment 5) presented 20% of plants without affection, compared to the preventive treatment with 2′,4′ dihydroxychalcone (Treatment 3) which presented 60% of plants who did not present visual symptoms of the disease.

While this invention has been described under the modalities indicated above, it might seem evident that other alternatives, modifications or variations would deliver the same results, however, we have been able to establish that the subject matter described in this application is essential for the success of the invention that It is described. Consequently, the embodiments of the invention are intended to be illustrative, not limiting. Various changes may be made without departing from the spirit and scope of the invention as defined in the following claims.

All patents, patent applications, scientific articles and other public documents that in the knowledge of the applicant constitute the state of the art, have been adequately cited in this application.

### REFERENCES

1. Vogler B, Cholewa L, Schmidt J, Setzer W. Pharmacology online 2006, 3, 850-855.
2. Cano, A., Espinoza, M., Ramos, C., Delgado, G. J. Mex. Chem. Soc. 2006, 50 (2), 71-75.
3. Delle Monache, F.; De Lima, O.G.; Mello, J.F.; Delle Monache, G.; Marini Bettolo, G.B. Gazz. Chim. Ital. 1973, 103, 779.
4. Yang H, Wang D, Tong L, Cai B.C., Chinese Pharmaceutical Journal 2008. 43 (5), 338-340.
5. Flnatyszyn, O., Ferraro, G., Coussio, D. Acta Farnz. Buenos Aires 15 (1): 21-8 (1996)
6. Muñoz, O., Peña, R., Ureta, E., Montenegro, G., Caldwell, C., Timmermann, B. Z. Naturforsch. 2001. 56c, 273-277 7. Di Carlo, G., Mascólo, N., Izzo, A. A., Capasso, F., Life Sci. 1999, 65, 337-353.
8. Jooseok Shin, Mi Gyeong Jang, Jin Chan Park, Young Do Koo, Ji Young Lee, Kyong Soo Park, Sung Soo Chung, Kwangyong Park. Antidiabetic effects of trihydroxychalcone derivatives via activation of AMP-activated protein kinase. Journal of Industrial and Engineering Chemistry, Volume 60, 2018, Pages 177-184.

1. A composition to control bacterial infections in plants, wherein comprises as active ingredient a compound selected from:
2′,4′-dihydroxy-3-(methyl-2-buten-1-yl) dihydrochalcone (1);
glabranin (2);
isochordine (3);
2′,4′-dihydroxychalcone (4);
7-Hydroxyflavanone (5);
chordine (6);
izalpinine (7);
2′,4′-dihydroxydihydrochalcone (8);
2′,4′-dimethoxydihydrochalcone (9); and
2′,4′,6′-trihydroxy chalcone (10).

2. A composition according to claim 1, wherein comprises an effective amount between 50 mg/L to 1000 mg/L of said compound.

3. A method to control bacterial infections in plants, wherein comprises applying an effective amount between 50 mg/L to 1000 mg/L, of the composition described in claim 1 to the bacteria to be treated.

4. The method according to claim 3, wherein the composition has antibacterial activity under in vitro and in vivo conditions.

5. The method according to claim 3, wherein the bacterial infections to be controlled in plant crops correspond to Pseudomnas syringae pv actinidiae (Psa) in Kiwi crops.

6. Use of a compound selected from: 2′,4′-dihydroxy-3-(methyl-2-buten-1-yl) dihydrochalcone (1), glabranin (2), isochordine (3), 2′,4′-dihydroxychalcone (4), 7-Hydroxyflavanone (5), chordine (6), izalpinin (7), 2′,4′-dihydroxydihydrochalcone (8), 2′,4′-dimethoxydihydrochalcone (9) and 2′,4′,6′-trihydroxy chalcone (10), wherein it is used to prepare an agrochemical composition to control infections in plant crops.

7. The use of claim 6, wherein the composition comprises an effective amount between 50 mg/L to 1000 mg/L, of a compound selected from: 2′,4′-dihydroxy-3-(methyl-2-butene-1-yl) dihydrochalcone (1), glabranin (2), isochordine (3), 2′,4′-dihydroxychalcone (4), 7-Hydroxyflavanone (5), chordine (6), izalpinin (7), 2′,4′-dihydroxydihydrochalcone (8), 2′,4′-dimethoxydihydrochalcone (9) and 2′,4′,6′-trihydroxy chalcone (10).

8. The use of claim 6, wherein the composition has antibacterial activity under in vitro and in vivo conditions.

9. The use of claim 5, wherein the infections are caused by microorganisms of the species Pseudomonas syringae pv actinidiae code 770 and 8.43 and other agronomically relevant bacteria.
